(19) United States
(12) Patent Application Publication
(10) Pub. No.: US 2022/0000409 A1
(43) Pub. Date: Jan. 6, 2022

(54) ON-EAR ELECTROENCEPHALOGRAPHIC MONITORING DEVICE

(71) Applicant: Hsin-Yin Chiang, Strasbourg (FR)

(72) Inventor: Hsin-Yin Chiang, Strasbourg (FR)

(21) Appl. No.: 17/227,355

(22) Filed: Apr. 11, 2021

Related U.S. Application Data
(60) Provisional application No. 63/048,144, filed on Jul. 5, 2020.

(51) Int. Cl. A61B 5/291; A61B 5/257; A61B 5/00; A61B 5/31; A61B 5/369

(52) U.S. Cl. CPC A61B 5/291; A61B 5/257; A61B 5/0006; A61B 2560/04; A61B 5/369; A61B 5/6817; A61B 5/31

(57) ABSTRACT

Embodiments of the present invention (herein referred to simply as “the invention”) comprise an EEG monitoring device worn on or around a user's ears. In some embodiments of the invention, the device comprises a flexible printed circuit containing EEG sensors, skin adhesives or adhesive sensors, and a flexible extension to position the sensor adjusting the user's head size. The device may be designed so that when worn by a user, the sensors are placed at specific points on a user's head in order to accurately capture electroencephalography signals. Said specific points may be one or more points of a 10-10 EEG system. The EEG sensors may comprise or may be made of an adhesive material.

This patent application claims the benefit of priority of U.S. Provisional Application No. 63/048,144 entitled “On-Ear (EEG) Electroencephalography Monitoring Device” filed Jul. 5, 2020, which is hereby incorporated herein by reference in its entirety.

### BACKGROUND OF INVENTION

The present invention, herein referred to as “the invention,” relates to devices for monitoring, detecting, and processing electroencephalographic (EEG) signals of the human brain. More specifically, the invention discloses an instant and discrete EEG monitoring device or adapter that can be worn on or around a user's ears.

Generally speaking, electroencephalography is a monitoring method that records the electrical activity of the brain. Electroencephalography comprises measuring the brainwaves noninvasively via electrodes/sensors placed on the scalp and helps to establish an accurate diagnosis of brain activity. In neurology, one of the common diagnostic applications of electroencephalography is in diagnosing epilepsy. For patients with epilepsy, it is crucial for medical professionals to detect the unusual electrical activity in the brain when a seizure is triggered. When the patients do not experience a seizure the brain activity may remain normal. This means that unless the patients experience a seizure during an EEG recording, the doctor cannot diagnose the type of seizure in full confidence. Due to the unpredictable occurrence of seizures and the limited consulting duration per patient, e.g. a session of electroencephalography in hospital, there is indeed an urgent need for a portable, inconspicuous, and wearable EEG device that can be continuously worn by the patients throughout the day to overcome the current constraints of laboratory-based or hospital-based EEG tests.

Likewise, EEG monitoring also facilitates the efficiency of medical treatment of mental disorders, and the investigation of Parkinson's and Alzheimer's diseases. Several studies show that EEG signals can be used to determine the mental status, emotions, and moods of a user, and it has been applied to diagnose the mental disorders of patients. The detection of emotional profile via EEG signals is particularly important as it reflects the symptoms of mental disorders in the early stage and can be used to derive the patient's mood pattern (i.e. mood tracking), tracking the efficacy of the designated treatment accurately. In addition, the patient's emotional triggers can be found and further resolved with the professional's help to improve the quality of life of the patient. At the moment, mood tracking is done manually by the patient to log at fixed time slots. It often lacks accuracy as it relies on memories to recall the moods throughout a day. Furthermore, the act of recalling negative feelings may aggravate the mental status of the patient. The application of EEG monitoring can resolve these inconveniences by providing automatic mood tracking. To achieve mood tracking for a long duration throughout the day, having an inconspicuous EEG monitoring device is essential for the field of mental health.

Alzheimer's disease (AD) is a neurodegenerative disorder that is characterized by cognitive deficits that result in the reduced capacity of patients in daily life and behavioral disturbances. EEG has been demonstrated as a reliable tool in the research of AD and the diagnosis as it contributes to the differential diagnosis and the prognosis of the disease progression. Additionally, such recordings of EEG signals can add important information related to drug effectiveness. Similarly, electroencephalography has been proven to be necessary for efficiently managing Parkinson's disease. A portable and inconspicuous EEG device can facilitate the monitoring of these diseases without causing additional disturbances in daily life.

For all of these diseases and many more, effective monitoring of brain activities via electroencephalography for a long duration is essential. This is because certain health information such as the occurrence of emotional triggers and other abnormalities of electrical activity in the brain does not only take place exclusively in hospitals, in laboratories, or in private, but anytime and anywhere. Various systems for monitoring EEG signals have been known for several years and with the general technological development, EEG monitoring systems, which may be worn continuously by a person to be monitored, have been devised. However, the existing prior art EEG devices have bulky and obvious structures that can be worn exclusively in laboratories or in private.

In the existing prior art, several efforts have been made to provide an EEG monitoring device with one or more sensors integrated as a part of earwear, for example, US20190053766, KR101579364, and so on. One issue with such systems is that the EEG monitoring devices described in the prior art are not size adjustable, and therefore may not be appropriate for all users. Another issue with such systems is that many of the EEG monitoring devices described in the prior art are bulky and thus impractical for wearing inconspicuously throughout the duration of a user's day.

In order to overcome these aforementioned issues, the inventor herein proposes a device that may be worn on or around a user's ear inconspicuously, that is easily size-adjustable, and that is able to collect resourceful electroencephalographic information of users. The device proposed by the inventor may comprise compact components and be designed to be worn comfortably and inconspicuously throughout the duration of a user's day.

### SUMMARY OF INVENTION

Embodiments of the present invention (herein referred to as “the invention”) comprise an EEG monitoring device worn on or around a user's ear. In some embodiments of the invention, the device comprises a flexible printed circuit (FPC) containing EEG electrodes, herein referred to as “sensors”. Some embodiments of the invention further comprise electrooculography (EOG) sensors in addition to the EEG sensors. The FPC may be made of but not limited to Polyethylene terephthalate (PET) and Polyimide (PI). The device may be designed so that when worn by a user, the sensors are placed at specific points on a user's head in order to accurately capture electroencephalography signals. Said specific points may be one or more points of a 10-10 or 10-20 EEG system.

The invention may comprise further features such as adhesives between the sensors and the user's head. Said adhesives may be a pure adhesive that removably bonds the sensors to the user's skin. Other embodiments of the invention comprise adhesive sensors such as a conductive hydrogel which itself can work as both an adhesive and a sensor.

Embodiments of the invention may comprise a flexible material overmolded onto the FPC. Said flexible material may be a flexible material including but not limited to thermoplastic polyurethane (TPU), silicone rubber, nitrile rubber, and other synthetic rubbers. In embodiments of the invention, the FPC acts as an electrical circuit for the device. The FPC is able to limit the overall cross-sectional dimension of the device by working as a substrate to house the sensors. In other devices that exist in the art, EEG sensors made of polymers are housed by a metallic substrate that is afterwards soldered into or mechanically in contact with the rest of an electrical connection. To attach the sensor on this metallic insert, the minimum thickness including the metallic substrate is around 3-6 mm due to the processing constraints and the sensor will be rigid due to the metallic substrate. By utilizing an FPC, the combined thickness of a sensor and an electrical connection of the present invention can be 1-2 mm or even lower. This allows the overall device to be slim, flexible, and inconspicuous. This fact is particularly beneficial to the proposed embodiment as a portable EEG measuring device as the lighter design can assure the maximum comfort and the inconspicuousness of the product when in use, further enhancing user engagement on brainwave detection.

### BRIEF DESCRIPTION OF DRAWINGS

FIG. 1 illustrates the placement of sensors in a 10-10 EEG system.

FIG. 2 illustrates the placement of sensors in a 10-20 EEG system.

FIG. 3 illustrates an embodiment of the invention.

FIG. 4 illustrates a section of FPC overmolded by an elastic material.

FIG. 5 illustrates an embodiment of the invention worn by a user.

FIG. 6 illustrates a close-up view of an embodiment of the invention.

FIG. 7 illustrates a section of FPC with sensors attached.

FIG. 8 illustrates a section of FPC under an applied force.

FIG. 9 illustrates the size-adjustable nature of an FPC.

FIG. 10 illustrates the size-adjustable nature of an FPC in an embodiment worn by a user.

FIG. 11 illustrates the electrical components of an EEG processing unit.

FIG. 12 illustrates the flow of EEG data between the EEG device and other devices.

### DETAILED DESCRIPTION

The description of the invention provided herein is for exemplary purposes and is not intended to limit the invention to any of the embodiments described herein. The figures used to support this specification are not intended to limit the invention to any specific shape, size, aesthetic design, or any other feature or property of the invention. The claimed invention is best understood by the appended claims.

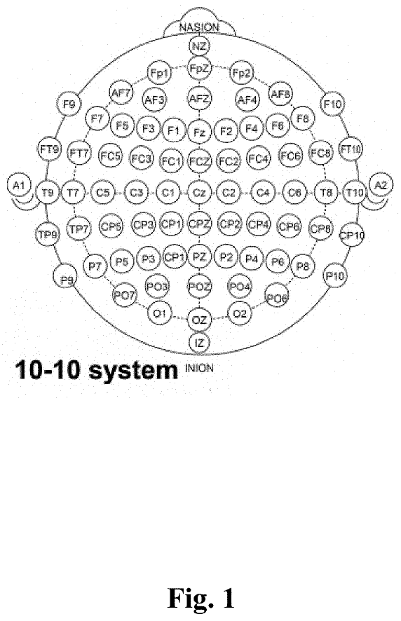

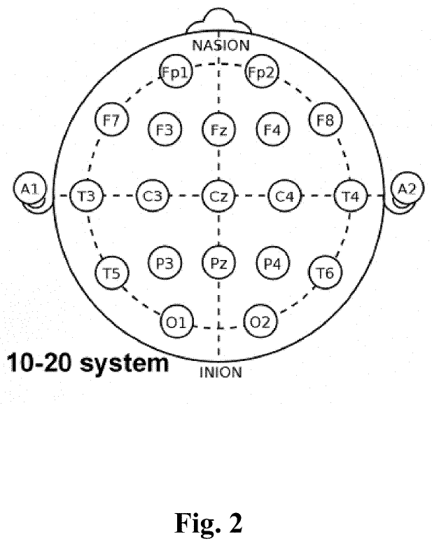

There are two systems of standardized EEG locations which are the 10-10 EEG system and the traditional 10-20 EEG system. FIG. 1 illustrates the placement of electrodes (sensors) on a user's head in the 10-10 EEG system which has been referred to in the present invention. FIG. 2 illustrates the placement of electrodes (sensors) on a user's head in the 10-20 EEG system.

FIG. 1 illustrates a 10-10 system which is internationally recognized and ensures the placement of EEG sensors to be standardized for further analysis. The 10-10 system of FIG. 1 is derived by modifying the conventional 10-20 system (as shown in FIG. 2) and is referred to as Modified Combinatorial Nomenclature (MCN). As illustrated in FIG. 2, the conventional system divides the scalp into 10% or 20% of the total front-back or right-left distance of the skull, and therefore the sensor spacing is proportional to the skull. EEG sensors are placed to cover all the brain regions which are broadly categorized as Frontal (F), Temporal (T), Parietal (P), and Occipital (O). The numbering system is odd numbers to the left side and even numbers to the right side, while z for ‘zero’ represents the midlines numbers. The 10-10 system shown in FIG. 1 uses 1, 3, 5, 7, 9 for the left hemisphere which represents 10%, 20%, 30%, 40%, 50% of the inion-to-nasion distance, respectively. Compared to the 10-20 system of FIG. 2, in the 10-10 system of FIG. 1, the new letter codes are applied. The introduction of extra letter codes allows the naming of intermediate sensor sites, namely: AF—between Fp and F, FC—between F and C, FT—between F and T, CP—between C and P, TP—between T and P, and PO—between P and O, Also, the 10-10 system renames four sensors of the 10-20 system: T3 becomes T7, T4 becomes T8, T5 becomes P7, and T6 becomes P8.

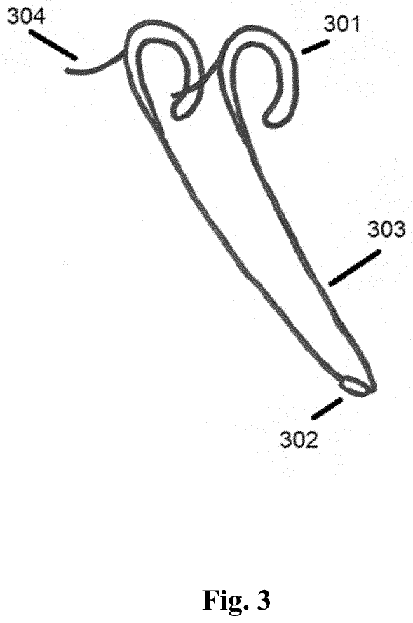

The invention comprises an on-ear electroencephalographic (EEG) monitoring device that comprises sensors that detect EEG signals from human brains, following the 10-10 EEG system. The invention may be worn on or around a user's ears so that the sensors are placed in the appropriate places on the user's head. FIG. 3 illustrates an embodiment of the invention that comprises one or more ear loops 301. Each ear loop may comprise a flexible printed circuit (FPC) which contains one or more sensors and a flexible material overmolded on top of the FPC. The flexible printed circuits may be connected to an EEG signal processing unit 302 by an electrical wire 303. In other embodiments of the invention, the EEG signal processing unit may be directly concealed in one or more ear loops 301 with the flexible printed circuits directly connected to the EEG signal processing unit.

Some embodiments of the invention comprise a flexible extension 304 that extends from the ear loop 301. The flexible extension may be a part of the FPC of the ear loops 301, and may also comprise sensors and a flexible material overmolded on top of the FPC. The purpose of the flexible extensions is to house sensors that contact the FT9/FT10 points of the user's head. Said FT9/FT10 points of the 10-10 EEG system are described further herein.

The FPC may provide the electrical connectivity between the EEG sensors. The FPC allows direct sensor attachment to ensure a minimum thickness/dimension of the earwear. The FPC may be made of a material including but not limited to polyimide (PI) and polyethylene terephthalate (PET).

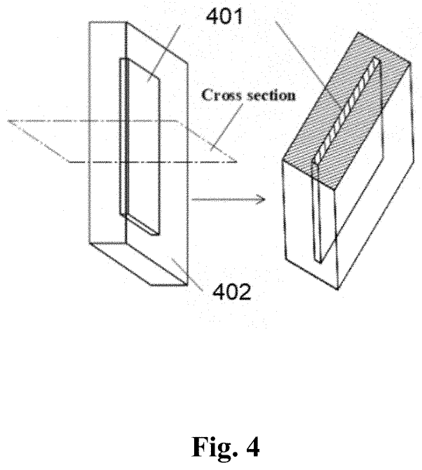

During the manufacturing process of the proposed earwear, the FPC with the sensors attached will be overmolded (using injection molding, compression molding, or similar molding processes) by an elastic material. FIG. 4 illustrates the configuration of materials in overmold where a material, in this case a FPC, is embedded within another material(s), here referring to an elastic material. This is to say that the elastic material will cover the main part of FPC and has openings at sensor locations to allow the sensors to come in contact with the user's head. This is to say that in some embodiments, the sensors may come into account with a user's head, while the FPC is completely covered by the sensors and elastic polymer(s).

The elastic material, also referred to herein as the “flexible material”, may comprise a material such as a flexible plastic including but not limited to thermoplastic polyurethane (TPU), silicone rubber, nitrile rubber, and other synthetic rubbers. Said material is used to form the shape of the ear loops. The FPC is overmolded within said material in order to provide the electrical connection and sensor housing for the device. The resulting combination of FPC and flexible material is flexible and bendable.

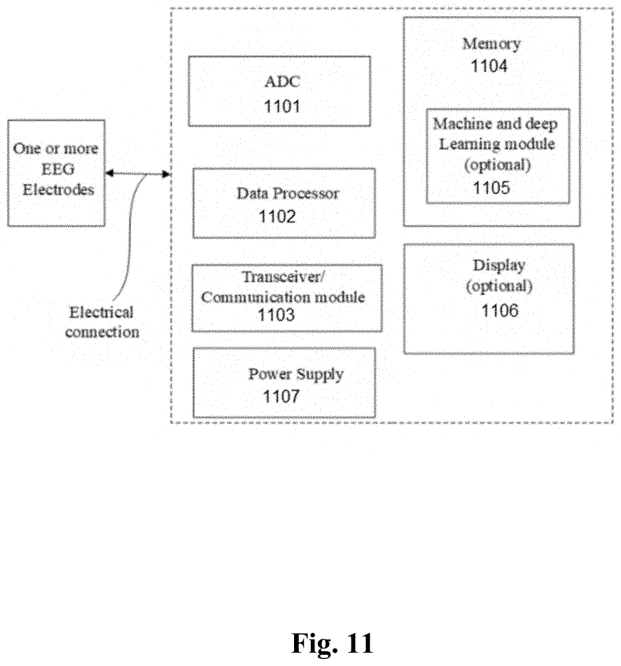

The EEG processing unit 302 may comprise a housing that houses an electronics unit. The electronics unit preferably comprises an analog-to-digital converter (ADC), a data processor, a transceiver/communication module, a memory, a machine and deep learning module embedded or stored in the memory, a display, and a power supply as shown in FIG. 11.

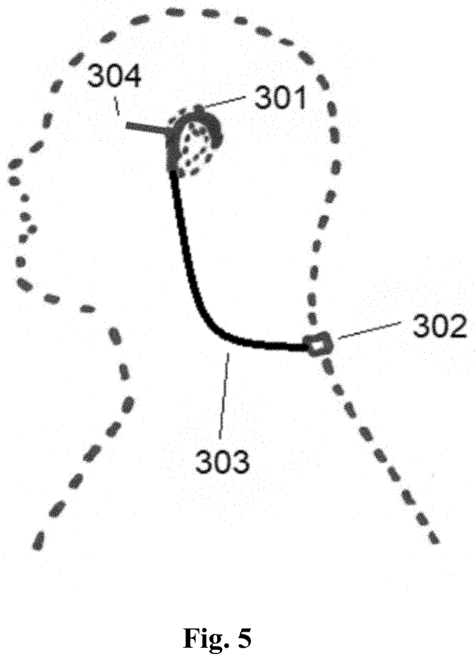

FIG. 5 illustrates the embodiment of the invention from FIG. 3 worn by a user. One of the one or more ear loops 301, the EEG signal processing unit 302, at least a portion of the wire 303, and one flexible extension 304 are visible in FIG. 5. In the embodiment illustrated in FIG. 5, the EEG signal processing unit may rest below the user's neck when the invention is worn by a user. In other embodiments, the EEG signal processing unit may be located at any other point on a user's head, neck, or body when the invention is worn by a user.

Also as illustrated in FIG. 5, the ear loops may be worn around a user's outer ears so that they contact the user's head and allow for proper placement of the EEG sensors on the user's head. The FPC material of the ear loops is flexible and adjustable in order to fit the user's head. In this manner, the invention is an improvement over the prior art in that it is easily size-adjustable by a user. Furthermore, skin adhesives may be applied on the embodiment, as the contour of the ear loop 301 and the extension 304, to fix the embodiment in place. The material of the skin adhesive may include but not limited to pressure sensitive adhesive (PSA) and biomimetic adhesive. Such an implementation of skin adhesive is able to ensure optimized signal acquisition from motion artifacts and coherent signal interpretation of EEG data. Said adhesives are described further herein.

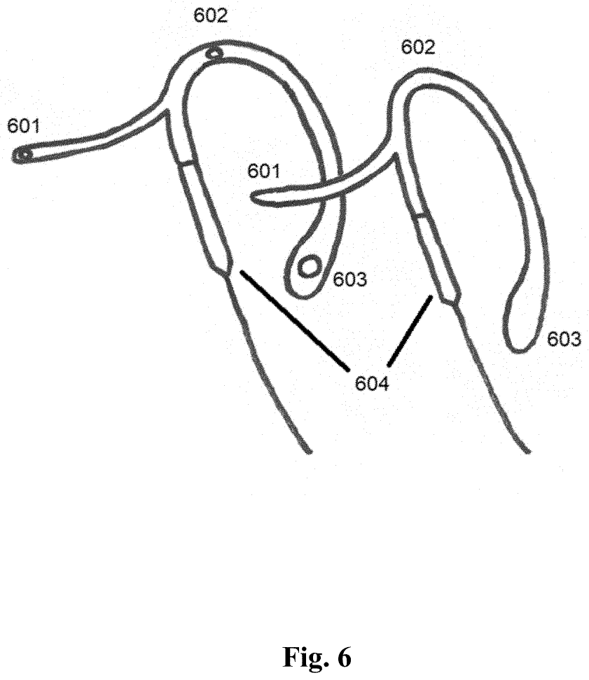

FIG. 6 illustrates a close-up view of the invention from FIG. 3. In FIG. 6, two ear loops are visible with three sensor locations designated by circles. The circles in FIG. 6 are only visible on one of the two illustrated ear loops to show that the sensors in some embodiments of the invention are only visible on the face of the ear loops that contact the user's head. Therefore, in the embodiments illustrated in FIG. 6, the ear loops are illustrated at the angle in which only the sensors of one ear loop are visible.

The sensor locations illustrated in FIG. 6 are Sensor Location A 601, Sensor Location B 602, and Sensor Location C 603. The sensor locations may match up with specific EEG points of a user's head. Said points may be FT9/FT10 for Sensor Location A 601, T9/T10 for Sensor Location B 602, and A1/A2 for Sensor Location C 603, according to the 10-10 system shown in FIG. 1. Sensor Location A 601 may be located on the flexible extensions 304. Also visible in FIG. 6 are soldering positions 604 that connect the FPC to the electrical wire where the solderings are concealed inside the ear loop 301. In some embodiments of the invention, the solderings are also overmolded by the same flexible material that is overmolded onto the FPC. In some embodiments, the ear loop 301 may equip additional sensor(s) for EEG or electrooculography (EOG) signal acquisition or as a bias input of the EEG signal processing unit.

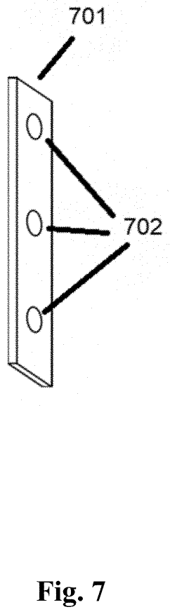

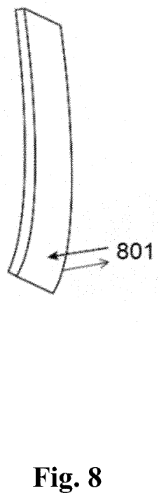

The application of FPC assures EEG signal quality by providing flexibility and adjustability. An advantage of using an FPC, instead of electrical wires with attached sensors, is that the FPC/sensor combination, i.e. direct attachment of sensors onto a FPC, is thin and flexible unlike traditional methods using rigid substrates carrying sensors. Therefore, the FPC/sensor combination can adapt to different user' head sizes. FIG. 7 illustrates a section of a FPC 701 with attached sensors 702. Furthermore, the FPC has a high restoring force perpendicular to its sheet-like plane even after overmolding, which enables an adequate pressure from a pre-formed FPC applied on the sensors toward the skin. As shown in FIG. 8, under an applied force, the FPC is able to restore to its original form after the release of force, annotated as the double arrows 801. The FPC permits flexibility to the device and movement exclusively in the direction perpendicular to the FPC's sheetlike surface plane, that is, in the sagittal/longitudinal plane of the user. With these physical properties of FPC, each sensor such as Sensor Location A 601 in FIG. 6 on the extension of ear loop as described further herein can be adjusted for better contact of the EEG sensor therein onto the designated 10-10 positions, for example Sensor Location A 601 to FT9/FT10, in terms of signal quality and comfort by adapting to the head size of the user.

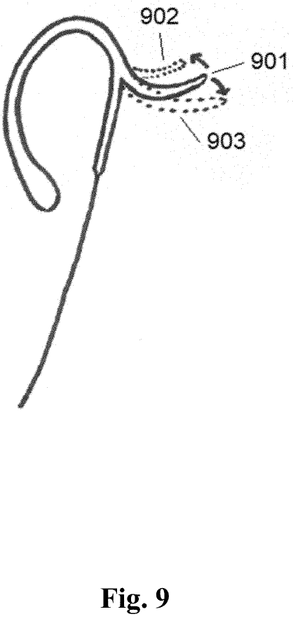

FIG. 9 illustrates the flexible extension (304 in FIG. 3) of the device in 3 positions, a first position 901 (on the surface plane), a second position 902 (into the plane/page), and a third position 903 (out of the plane/page). Arrows are illustrated to show the permissible movement of the flexible extension 304 between these 3 positions in the sagittal/longitudinal plan of the user.

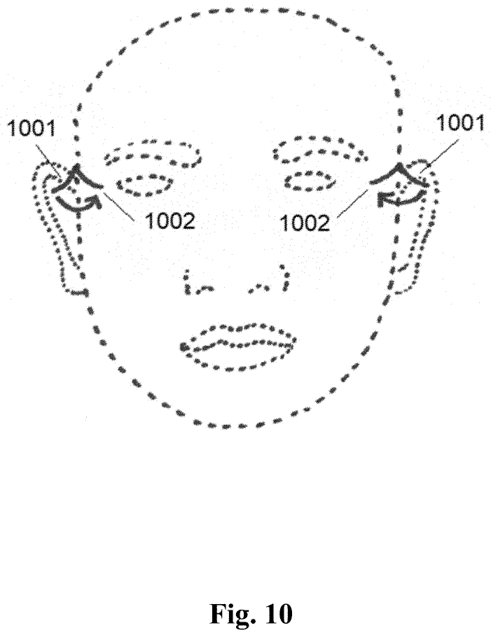

FIG. 10 illustrates a user's head that is wearing the EEG device. The flexible extensions 304 are visible in 2 positions, a first position 1001 and a second position 1002. As illustrated by the arrow showing the movement from the first position 1001 to the second position 1002, the flexible extension, an elastic material overmolding on a preformed FPC, is adjustable to adapt the size of user's head with an adequate pressure from the restoring force in order for the sensors located on the flexible extensions to come in contact with the user's head. Similarly, the FPC may also permit the sensor 603 in FIG. 6 located on the ear loops to be adjustable and fit on the individual's mastoid, according to the user's head size. In this way, the flexible nature of the FPC allows the device to be size-adjustable.

The invention may comprise one or more skin adhesives between the sensors and the user's head. The adhesive may serve to secure the invention to the user's head while in use. The adhesive may further serve to stabilize the EEG signal and minimize movement artifacts that hamper EEG analysis. The skin adhesive can be used repeatedly, to minimize the waste consumption of use, and can be cleaned easily with water due to its property of hydrophobicity, ensuring the user's hygienic condition. The skin adhesive may be either a pure adhesive or adhesive sensors.

In the embodiments of the invention in which the adhesive is a pure adhesive, said adhesive may be a pressure sensitive adhesive (PSA). This type of adhesive is non-conductive and located near the sensors. It exists solely to secure the sensors to the user's head. Alternatively, in the embodiments of the invention in which the adhesive is a pure adhesive and located near the near sensors, said adhesive may be a non-conductive biomimetic adhesive such as but not limited to “gecko tape”or “octopus tape”. This type of adhesive comprises microstructures that mimic the fibres of a gecko's feet or octopus' suction cups in order to secure the device to the user's head.

In the embodiments of the invention in which the adhesive is an adhesive sensor, said adhesive may be a conductive hydrogel. This is a conductive material that may serve as both the sensors and the adhesive. Alternatively, in the embodiments of the invention in which the adhesive is an adhesive sensor, said adhesive may be a conductive biomimetic adhesive. The conductive biomimetic adhesive is similar to the non-conductive biomimetic adhesive described herein, except that the conductive biomimetic adhesive is made of a conductive material and therefore may serve as both the sensors and the adhesive.

Various embodiments of the invention may comprise sensor locations that correspond to the 10-10 EEG system. The locations of the EEG sensors on the user's head are crucial to provide detailed information of electrical activities in the user's brain. The specific EEG sensor locations described herein (FT9, FT10, T9, T10, A1, and A2) are the designated positions in the 10-10 EEG system and are able to form at least 10 channels, said 10 channels being FT9-FT10, FT9-T9, FT9-T10, FT9-A1, FT10-T9, FT10-T10, FT10-A1, T9-T10, T9-A1, and T10-A1. These channels are able to detect various brainwaves such as alpha, beta, and gamma waves as well as to provide the correlation levels for designated applications such as emotion recognition.

For example, the channel T9-T10 is able to provide the correlation of neutral-positive emotions via analyzing alpha wave, FT9-T9 for the correlation of negative-neutral emotions via both alpha and beta waves, and FT10-T10 for phase synchronization of negative-positive emotions via beta wave. Furthermore, these channels are capable of detecting different statuses of a user's brain as arousal and valence levels. FT9-A1 and FT10-A2 are able to provide EOG signals that can be used for facial expression recognition. For standard EEG acquisition, it is essential to have the reference points such as A1 and A2 to obtain local bio-potentials at individual EEG positions.

In operation, the EEG sensors configured on the FPCs acquire EEG signals from the user's brain at any instant of time and send said EEG signals to the EEG processing unit. FIG. 11 illustrates the electronic components of the EEG processing unit in some embodiments of the inventions. Once the EEG signals have been measured by the EEG sensors, the captured EEG signals in the analog form are transmitted to an ADC 1101 of the EEG processing unit. The ADC 1101 converts analog EEG signals from the user to a digital form which is then communicated to a data processor 1102. The data processor 1102 includes one or more processors/microcontrollers known in the art and conducts signal analysis. This signal analysis by the processor may be powered by machine learning or deep learning algorithms 1105 that may be optionally stored in the memory 1104 unit to distinguish if the captured signal by the EEG sensors is within the acceptable EEG domain (e.g. frequency, amplitude). The machine learning or deep learning algorithms 1105 can be updated regularly via wired connection or via wireless communication using transceiver/communication module 1103. After the analysis is done by the processor, the result may then be displayed on a display unit 1106 of a device such as a smartphone, tablet, general purpose computer, or special purpose computer. The EEG data from the electronics unit is preferably transmitted using wireless technologies including but not limited to WiFi and Bluetooth. The EEG data is transmitted by the transceiver/communication module 1103. The power supply 1107 is adapted to power the various components of the EEG processing unit.

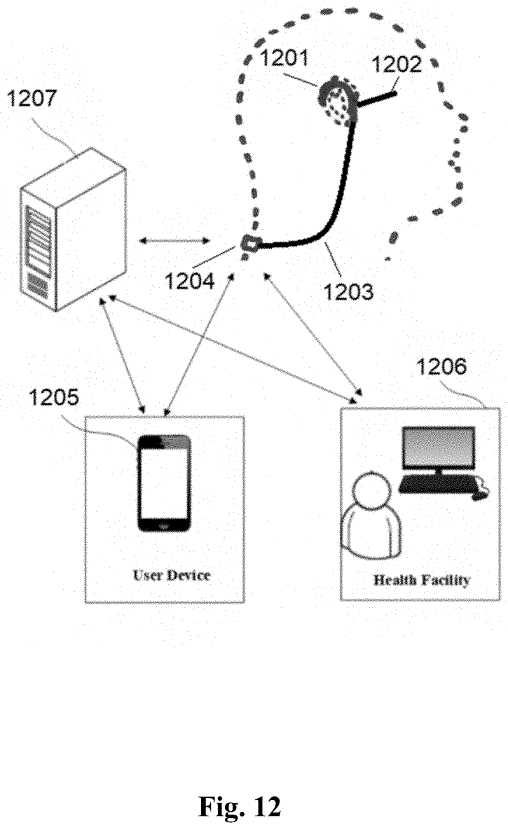

FIG. 12 illustrates a possible flow of EEG data between the device (one example embodiment), a server or database 1207, a user device 1205, and a health facility 1206. EEG data is gathered from the sensors on the extension 1202 and the ear loop 1201 and is transmitted to the EEG processing unit 1204 via the electrical connection 1203. In some embodiments, the EEG processing unit and the electrical connection are concealed within the ear loop 1201. Said data may then be sent to a server or database 1207 to be stored. The data may then be sent from the server or database 1207 to a user device 1205 or health facility 1206. Alternatively, the EEG data may be sent directly to the user device such as but not limited to smartphone, tablet, special purpose computer, or general purpose computer 1205 or health facility 1206 from the EEG processing unit 1204. The user device 1204 may be enabled to generate corresponding EEG data in various formats (.csv, .mat, .xlxs, etc.) to facilitate communication with medical professionals. In some embodiments, the health facility 1206 may be any facility that treats patients. The health facility 1206 may comprise its own servers, databases, general purpose computers, special purpose computers, or other devices used to receive and process the EEG data sent from the EEG processing unit 1204 or the server or database 1207.

What is claimed is:

1. An electroencephalographic device comprising:
one or more flexible printed circuits;
one or more electroencephalographic sensors;
an electroencephalographic processing unit; and
a connection between the one or more flexible printed circuits,
wherein the electroencephalographic device is worn around a user's ear(s) and contacts one or more points on the user's head.

2. The electroencephalographic device of claim 1, wherein the electroencephalographic processing unit comprises:
a data processor;
a wireless data transmitter;
a configured power source; and
a memory storage component.

3. The electroencephalographic device of claim 1, further comprising a flexible material overmolded onto the one or more flexible printed circuits.

4. The electroencephalographic device of claim 1, further comprising an adhesive to ensure contact with the user's head.

5. The electroencephalographic device of claim 4, wherein the adhesive is a pressure sensitive adhesive.

6. The electroencephalographic device of claim 4, wherein the adhesive is a non-conductive biomimetic adhesive.

7. The electroencephalographic device of claim 1, wherein the user may adjust the size of the device by bending the one or more flexible printed circuits.

8. The electroencephalographic device of claim 1, wherein the number of one or more electroencephalographic sensors is at least 6, and at least some of said electroencephalographic sensors contact the user's head at points FT9, FT10, T9, T10, A1, and A2 as they exist in a 10-10 electroencephalography system.

9. The electroencephalographic device of claim 1, wherein the number of one or more electroencephalographic sensors is at least 4, and at least some of said electroencephalographic sensors contact the user's head at points FT9, FT10, T9, and T10 as they exist in a 10-10 electroencephalography system.

10. The electroencephalographic device of claim 1, wherein the number of one or more electroencephalographic sensors is at least 4, and at least some of said electroencephalographic sensors contact the user's head at points T9, T10, A1, and A2 as they exist in a 10-10 electroencephalography system.

11. The electroencephalographic device of claim 1, further comprising one or more electrooculographic sensors for analyzing information such as facial expression.

12. An electroencephalographic device comprising:
one or more flexible printed circuits;
one or more adhesive electroencephalographic sensors;
an electroencephalographic processing unit; and
a connection between the one or more flexible printed circuits and the electroencephalographic processing unit,
wherein the electroencephalographic device is worn around a user's ear(s) and contacts one or more points on the user's head.

13. The electroencephalographic device of claim 12, wherein the electroencephalographic processing unit comprises:
a data processor;
a wireless data transmitter;
a configured power source; and
a memory storage component.

14. The electroencephalographic device of claim 12, further comprising a flexible material overmolded onto the one or more flexible printed circuits.

15. The electroencephalographic device of claim 12, wherein the user may adjust the size of the device by bending the one or more flexible printed circuits.

16. The electroencephalographic device of claim 12, wherein the number of one or more adhesive electroencephalographic sensors is at least 6, and at least some of said adhesive electroencephalographic sensors contact the user's head at points FT9, FT10, T9, T10, A1, and A2 as they exist in a 10-10 electroencephalography system.

17. The electroencephalographic device of claim 12, wherein the number of said adhesive electroencephalographic sensors is equal to or more than 4, and at least some of said adhesive electroencephalographic sensors contact the user's head at points FT9, FT10, T9, and T10 as they exist in a 10-10 electroencephalography system.

18. The electroencephalographic device of claim 12 wherein the number of one or more adhesive electroencephalographic sensors is at least 4, and at least some of said adhesive electroencephalographic sensors contact the user's head at points T9, T10, A1, and A2 as they exist in a 10-10 electroencephalography system.

19. The electroencephalographic device of claim 12, further comprising one or more electrooculographic sensors for analyzing information such as facial expression.

20. The electroencephalographic device of claim 12, wherein the adhesive sensors are made of conductive hydrogel.

21. The electroencephalographic device of claim 12, wherein the adhesive sensors are made of a conductive biomimetic adhesive material.
